(19) United States
(12) Patent Application Publication
(10) Pub. No.: US 2023/0346310 A1
(43) Pub. Date: Nov. 2, 2023

(54) WEARABLE ELECTROMYOGRAPHY DEVICE

(71) Applicant: TAIWAN TEXTILE RESEARCH INSTITUTE, NEW TAIPEI CITY (TW)

(72) Inventors: Szu-Yu LIAO, NEW TAIPEI CITY (TW); Chien-Lung SHEN, NEW TAIPEI CITY (TW); Pai-En YANG, NEW TAIPEI CITY (TW); Mei-Chuan TSENG, NEW TAIPEI CITY (TW); Chieh YIN, NEW TAIPEI CITY (TW); Ching YEH, NEW TAIPEI CITY (TW)

(21) Appl. No.: 17/731,298

(22) Filed: Apr. 28, 2022

(51) Int. Cl. A61B 5/00; A61B 5/296; A61B 5/27

(52) U.S. Cl. CPC A61B 5/6804; A61B 5/296; A61B 5/27; A61B 2562/164

(57) ABSTRACT

A wearable electromyography device includes a garment, a plurality of fabric electrodes, and an insulating film. The garment has a plurality of anchor structures disposed on a first surface of the garment and a plurality of holes penetrating the garment. The fabric electrodes are disposed on the first surface of the garment. The insulating film is disposed on the first surface of the garment and seals peripherals of the fabric electrodes. A segment of the garment without being covered by the insulating film has a first elastic extension rate, a segment of the garment being covered by the insulating film has a second elastic extension rate, and the first elastic extension rate is greater than the second elastic extension rate.

### BACKGROUND

### Field of Invention

The present invention relates to a wearable electromyography device.

### Description of Related Art

In recent years, man-machine interaction integrates various brain-computer interface technologies such as virtual reality technology, motion sensing, biofeedback technology etc., which develops rapidly in the rehabilitation field. The method of using muscle electric signal feedback to realize man-machine interaction not only increases the participation interest of user on machine interactive interface, but also extracts useful information in complicated bioelectrical signals for the benefit of neuromuscular tracking and evaluating, and improves rehabilitation efficiency.

Conventional electromyography (EMG) devices are wearable devices and require firmly contacting the electrodes with user's skin. However, when the user exercise, sweat or extension and flexion motions of muscles would lead to fabric falling or stretch deformation, thus the electrodes are incorrectly positioned, and the accuracy of measured EMG signals are not reliable.

### SUMMARY

An aspect of the invention provides a wearable electromyography device. The wearable electromyography device includes a garment, a plurality of fabric electrodes, and an insulating film. The garment has a plurality of anchor structures disposed on a first surface of the garment and a plurality of holes penetrating the garment. The fabric electrodes are disposed on the first surface of the garment. The insulating film is disposed on the first surface of the garment and seals peripherals of the fabric electrodes. A segment of the garment without being covered by the insulating film has a first elastic extension rate, a segment of the garment being covered by the insulating film has a second elastic extension rate, and the first elastic extension rate is greater than the second elastic extension rate.

According to some embodiments, the first elastic extension rate is 130% to 160%.

According to some embodiments, the second elastic extension rate is 110% to 120%.

According to some embodiments, the holes are respectively disposed between the anchor structures.

According to some embodiments, the garment includes a first region and a second region, and a distribution density of the holes at the first region is greater than a distribution density of the holes at the second region.

According to some embodiments, the wearable electromyography device further includes a wrap fastener connected to the garment.

According to some embodiments, the wrap fastener includes a loop and hook fastener.

According to some embodiments, the wearable electromyography device further includes a plurality of conductive pieces disposed on the garment and connected to the fabric electrodes, respectively.

According to some embodiments, the wearable electromyography device further includes a wireless transmission module connected to the conductive pieces.

According to some embodiments, the wearable electromyography device further includes a plurality of fabric wirings connecting to the fabric electrodes to the conductive pieces, wherein the insulating film covers the fabric wirings.

According to some embodiments, the fabric electrodes include a first differential electrode pair, a second differential electrode pair, and a reference electrode.

According to some embodiments, the maximum static friction coefficient of the garment is from 0.45 to 0.50.

According to some embodiments, an area ratio of the holes to the garment is from 10% to 15%.

According to some embodiments, wherein the anchor structures include friction yarns knitted into the garment.

According to some embodiments, the friction yarns are knitted into the garment by flat knitting.

According to some embodiments, the friction yarns are knitted into the garment by warp knitting.

According to some embodiments, the garment includes a plurality of basic units and a plurality of linking yarns selectively interconnecting the basic units. Each of the basic units includes a plurality of elastic yarns, a plurality of friction yarns wound on the elastic yarns, and a plurality of floating weft yarns wrapped on an outer facing side of the friction yarns. The linking yarns selectively interconnect the basic units.

According to some embodiments, the holes are spaces between the basic units without being interconnected by the linking yarns.

According to some embodiments, the linking yarns interconnect the friction yarns of the basic units.

According to some embodiments, an inner facing side of the friction yarns is free of being wrapped by the floating weft yarns.

It is to be understood that both the foregoing general description and the following detailed description are by examples, and are intended to provide further explanation of the invention as claimed.

According to some embodiments of the invention, the wearable electromyography device is able to secure the electrodes at the correct positions thereby solving the problem of electrode shifting due to clothing falling or stretch deformation during the exercise. The accuracy of motion analysis using the wearable electromyography device can be improved accordingly.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a schematic view of the wearable electromyography device according to some embodiments of the invention;

FIG. 2 is a schematic view of a stretch state of the wearable electromyography device according to some embodiments of the invention;

FIG. 3A is an EMG signal graph of a user wearing the wearable electromyography device of an embodiment of the invention and doing exercise;

FIG. 3B is a comparative EMG signal graph of the user wearing another wearable electromyography device and doing exercise;

FIG. 4A is a front view of the wearable electromyography device of some other embodiments of the invention;

FIG. 4B is a schematic view of the user wearing the wearable electromyography device of FIG. 4A;

FIG. 5 is a front view of the wearable electromyography device according to yet other embodiments of the invention;

FIG. 6A is a knitting graph of the garment of the wearable electromyography device according to some embodiments of the invention;

FIG. 6B is a yarn loop schematic view of the garment of the wearable electromyography device according to some embodiments of the invention; and

FIG. 7A and FIG. 7B, which are knitting graphs of the garment of the wearable electromyography device according to some embodiments of the invention.

### DESCRIPTION OF THE EMBODIMENTS

Reference will now be made in detail to the present embodiments of the invention, examples of which are illustrated in the accompanying drawings. Wherever possible, the same reference numbers are used in the drawings and the description to refer to the same or like parts.

An aspect of the invention provides a wearable electromyography device. The wearable electromyography device is able to secure the electrodes at the desired positions thereby solving the problem of electrode shifting due to clothing falling or stretch deformation during the exercise. The accuracy of motion analysis using the wearable electromyography device can be improved accordingly.

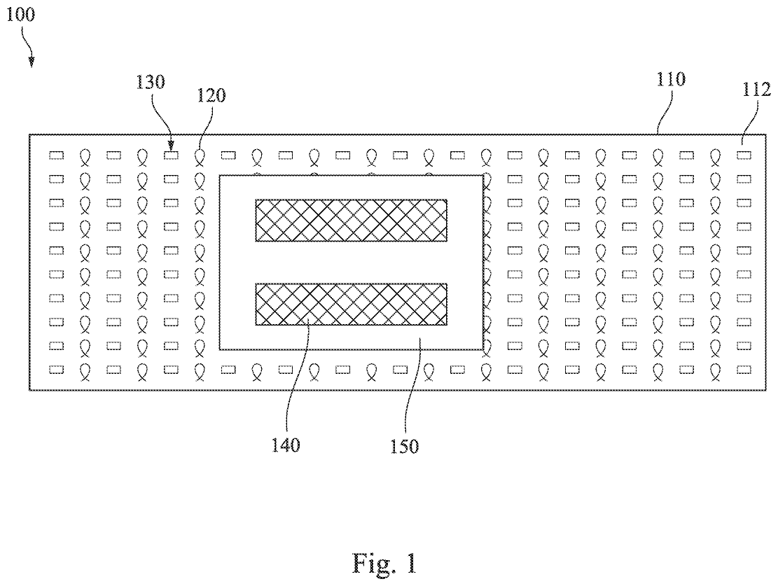

Reference is made to FIG. 1, which is a schematic view of the wearable electromyography device according to some embodiments of the invention. The wearable electromyography device 100 includes a garment 110, a plurality of fabric electrodes 140, and an insulating film 150. The garment 110 includes a plurality of anchor structures 120 disposed on a first surface 112 of the garment 110 and a plurality of holes 130 penetrating the garment 110. The fabric electrodes 140 are disposed on the first surface 112 of the garment 110. Specifically, the first surface 112 of the garment 110 is the surface touching user's skin when the user wears the wearable electromyography device 100 such that the fabric electrodes 140 are in contact with the user's skin to detect electromyography (EMG) signals. The insulating film 150 is disposed on the first surface 112 of the garment 110 and seals peripherals of the fabric electrodes 140. The segment of the garment 110 without being covered by the insulating film 150 has a first elastic extension rate, the segment of the garment 110 being covered by the insulating film 150 has a second elastic extension rate, and the first elastic extension rate is greater than the second elastic extension rate.

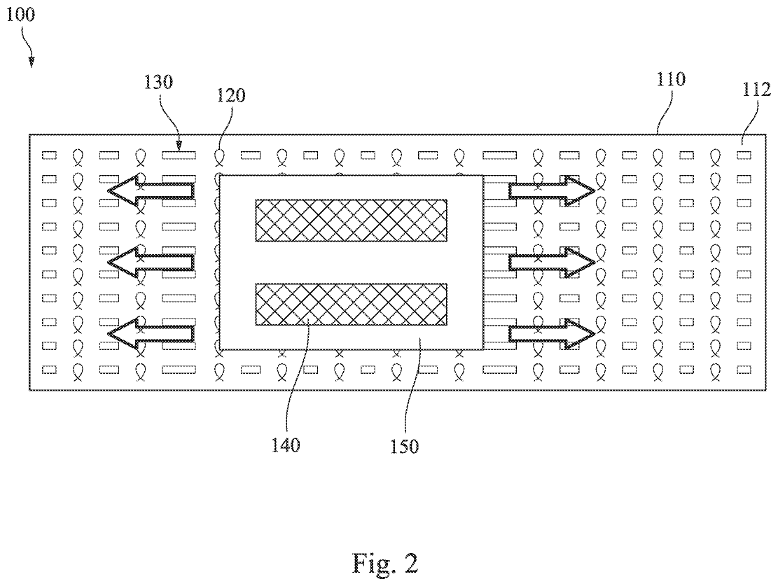

Reference is made to both FIG. 1 and FIG. 2, in which FIG. 2 is a schematic view of a stretch state of the wearable electromyography device according to some embodiments of the invention. When the user wearing the wearable electromyography device exercises, the extension and flexion of antagonist muscles cause the deformation of the garment 110, as shown in FIG. 2. The anchor structures 120 which are disposed on the first surface 112 of the garment 110 would secure on the user's skin to keep the relative position with the user's skin such that the wearable electromyography device 100 can be prevented from falling from the user's skin. In other word, even if the user's skin stretches because of extension and flexion muscle motions, the anchor structures 120 would still be secured on the relative position with the user's skin, and the garment 110 would generate equal deformation in the direction as the user's skin stretching.

According to some embodiments, the anchor structures 120 are structures having high static friction coefficient. For example, in an exemplary embodiment, the garment 110 having the anchor structures 120 is placed on a glass, in which the glass inclines 20 degrees, the size of the garment 110 is 10 cm×10 cm, and the weight on the garment 110 is 155 grams. The maximum static friction coefficient of the garment 110 is from 0.45 to 0.50. If the maximum static friction coefficient of the garment 110 is less than 0.45, the function of the anchor structures 120 grabbing the user's skin may be failed. If the maximum static friction coefficient of the garment 110 is greater than 0.50, user may feel uncomfortable when the user wears the wearable electromyography device 100. In some embodiments, the anchor structures 120 include friction yarns knitted into the garment 110.

When the anchor structures 120 grab the user's skin to allow the garment 110 deforming in the direction as the user's skin stretching, the holes 120 on the wearable electromyography device 100 are enlarged accordingly. Therefore, the garment 110 can be stretched more smoothly.

In some embodiments, the holes 130 are arranged between the anchor structures 120, respectively. As a result, when the garment 110 is stretched and the anchor structures 120 are secured on the same position of the user's skin, the holes 130 are forced to be enlarged to absorb the corresponding deformation as well. In some embodiments, the area ratio of the holes 130 to the garment 110 is from 10% to 15%. If the area ratio of the holes 130 to the garment 110 is less than 10%, the stretchable amount of the garment 110 may be insufficient. If the area ratio of the holes 130 to the garment 110 is greater than 15%, the wearable electromyography device 100 may not be able to contact the user's skin firmly. The area ratio of the holes 130 to the garment 110 mentioned above is measured at a static state, in which the wearable electromyography device 100 is not stretched by an external force.

Because the elastic extension rate of the segment of the garment 110 uncovered with the insulating film 150 is greater than the elastic extension rate of the segment of the garment 110 covered with the insulating film 150, the deformation ability of the peripheral of the fabric electrodes 140, which is covered with the insulating film 150, is limited by the poor elastic extension rate. Therefore, the deformation of the fabric electrodes 140 and the peripheral is not obvious when the garment 110 is stretched and deformed. The contact area and position between the fabric electrodes 140 and user's skin keep the same when the user wears the wearable electromyography device 100, thus the detecting error of using the wearable electromyography device 100 can be reduced.

In some embodiments, the original elastic extension rate of the garment 110 (e.g. the first elastic extension rate) is 130% to 160%, and the elastic extension rate of the garment 110 coated with the insulating film 150 (e.g. the second elastic extension rate) is 110% to 120%. The aforementioned elastic extension rate is measured by ASTM method D5035-09, and the elastic extension rate of the garment 110 coated with the insulating film 150 refers to the elastic extension rate after the insulating film 150 is hot pressed on the garment 110, as a whole. In some embodiments, the material of the insulating film 150 can be thermoplastic polyurethane (TPU).

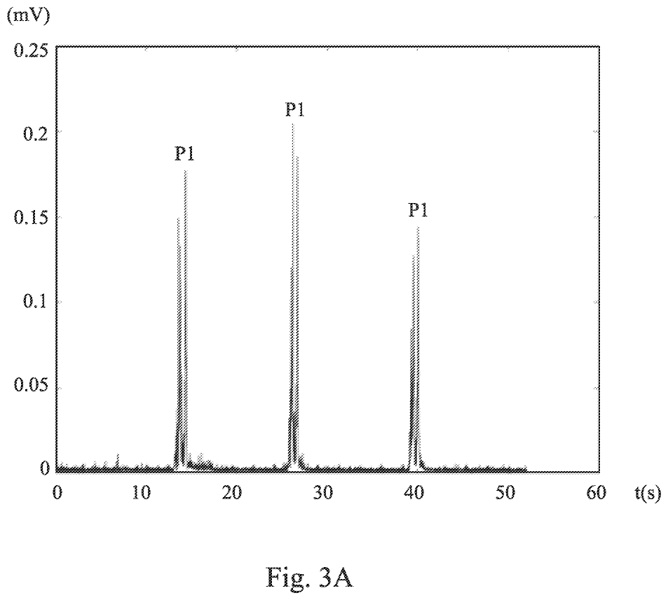

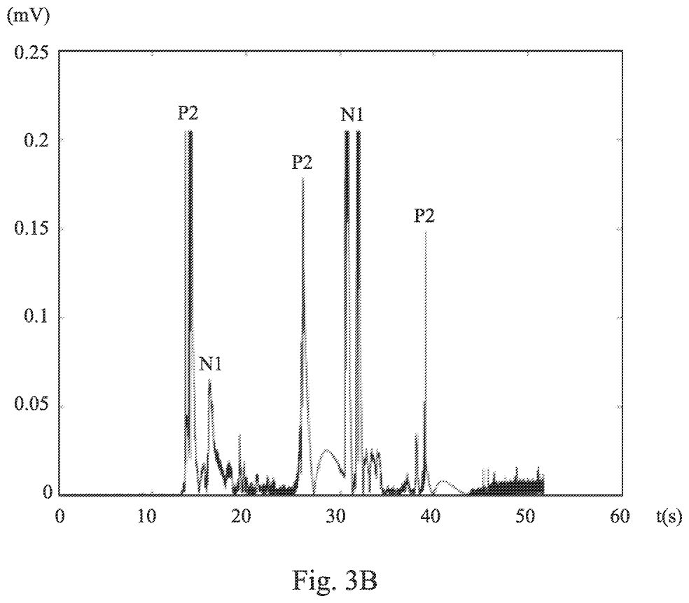

Reference is made to FIG. 3A and FIG. 3B, in which FIG. 3A is an EMG signal graph of a user wearing the wearable electromyography device of an embodiment of the invention and doing exercise, and FIG. 3B is a comparative EMG signal graph of the user wearing a wearable electromyography device having the same stereotype but without the anchor structures 120, the holes 130, and the insulating film 150.

These two wearable electromyography devices, having the same stereotype, are worn at the same position of the same user. The user then jumps every 10 seconds for three times. The measured EMG graphs are shown in FIG. 3A and FIG. 3B. As shown in FIG. 3A, the wearable electromyography device of the embodiment of the invention provides clear and obvious target features P1. On the contrary, as shown in FIG. 3B, the control wearable electromyography device not only provides target features P2 of jumping, but also provides noises N1, and the noises N1 are strong enough to affect the accuracy of the EMG measure result.

To sum up, by the design of the anchor structures, the holes, and the insulating film sealing the peripheral of the fabric electrodes, the fabric electrodes of the wearable electromyography device of the embodiments of the invention can stably and firmly contact the same position of the user's skin when the user exercises. Thus the measure accuracy of the wearable electromyography device of the embodiments of the invention can be improved.

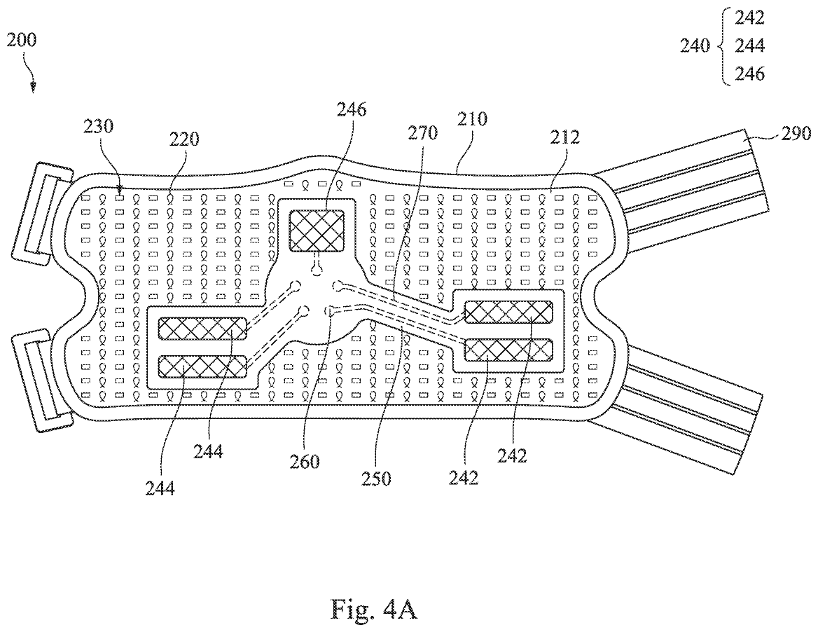

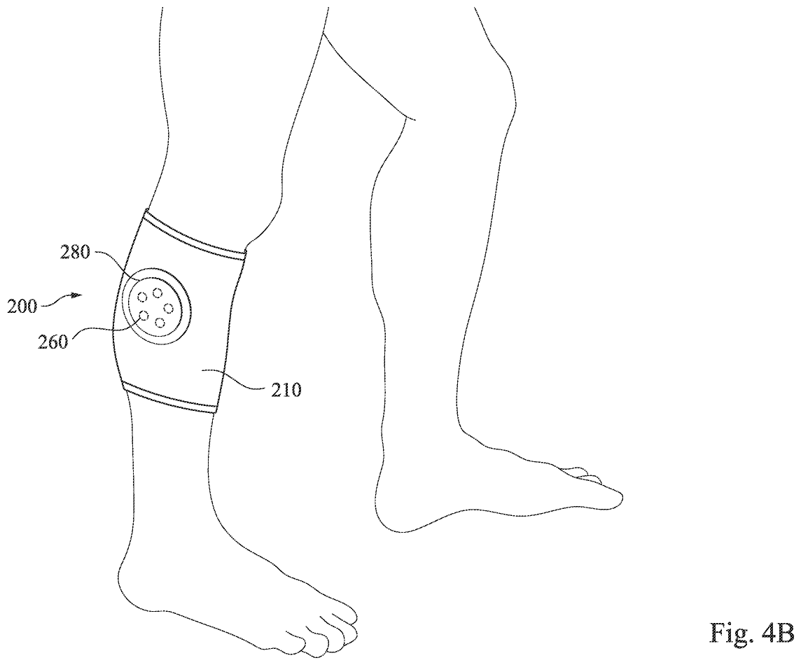

Reference is made to FIG. 4A and FIG. 4B, in which FIG. 4A is a front view of the wearable electromyography device of some other embodiments of the invention, and FIG. 4B is a schematic view of the user wearing the wearable electromyography device of FIG. 4A. The wearable electromyography device 200 includes a garment 210, a plurality of fabric electrodes 240, and an insulating film 250. The garment 210 includes a plurality of anchor structures 220 disposed on a first surface 212 of the garment 210 and a plurality of holes 230 penetrating the garment 210. The fabric electrodes 240 are disposed on the first surface 212 of the garment 210. The insulating film 250 is disposed on the first surface 212 of the garment 210 and seals peripherals of the fabric electrodes 240. The segment of the garment 210 without being covered by the insulating film 250 has a first elastic extension rate, the segment of the garment 210 being covered by the insulating film 250 has a second elastic extension rate, and the first elastic extension rate is greater than the second elastic extension rate.

In some embodiments, the fabric electrodes 240 include a first differential electrode pair 242, a second differential electrode pair 244, and a reference electrode 246. The first differential electrode pair 242 and the second differential electrode pair 244 are respectively disposed on the antagonist muscle pairs, and the reference electrode 246 is disposed on the position having less muscle motion, such as at the side surface of limbs. The shape of the electrodes of the first differential electrode pair 242 are elongated and are arranged parallel. The shape of the electrodes of the second differential electrode pair 244 are elongate and are arranged parallel. The axes of the first differential electrode pair 242 and the second differential electrode pair 244 are substantially perpendicular to the muscle direction, for better detecting EMG signals.

In some embodiments, the wearable electromyography device 200 includes a plurality of conductive pieces 260, a plurality of conductive wirings 270, and a wireless transmission module 280. The conductive wirings 270 connect the fabric electrodes 240 to the corresponding conductive pieces 260, and the wireless transmission module 280 is connected to the conductive piece 260. The EMG signals measured by the fabric electrodes 240 can be transmitted to the wireless transmission module 280 via the conductive wirings 270 and the conductive pieces 260, and the wireless transmission module 280 further transmits the EMG signals to other processing units for analysis.

In some embodiments, the conductive wirings 270 are sewed on the first surface 212 of the garment 210. In some other embodiments, the conductive wirings 270 are woven in the garment 210 by predetermined weaving processes. The insulating film 250 covers the conductive wirings 270 to seal the conductive wirings 270 between the garment 210 and the insulating film 250.

In some embodiments, the wearable electromyography device 200 further includes at least one wrap fastener 290 connected to the garment 210. After the wearable electromyography device 200 is positioned on the limb of the user, the wrap fastener 290 can surround the limb of the user to fix the wearable electromyography device 200 on the user. In some embodiments, the wrap fastener 290 is a self-bond fastener, such as a loop and hook fastener.

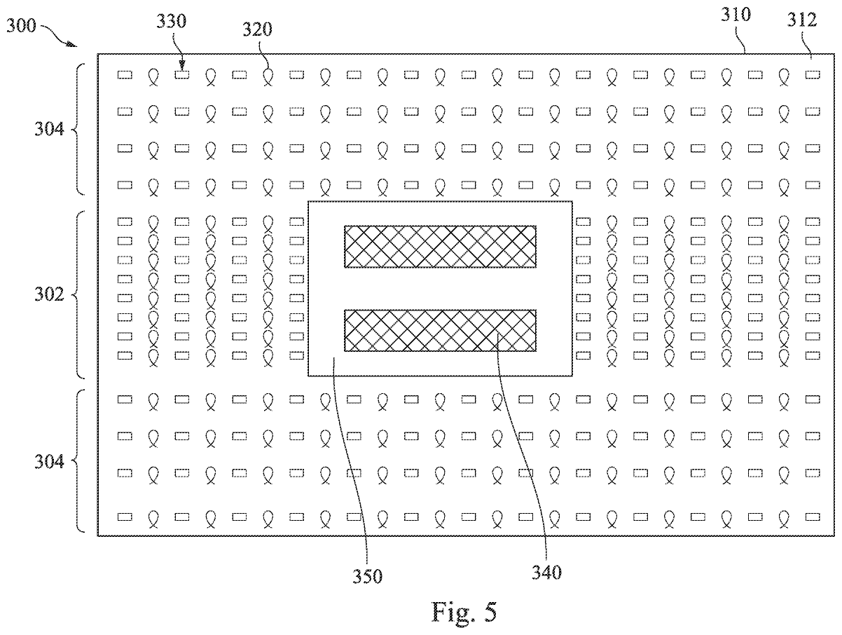

Reference is made to FIG. 5, which is a front view of the wearable electromyography device according to yet other embodiments of the invention. The wearable electromyography device 300 includes a garment 310, a plurality of fabric electrodes 340, and an insulating film 350. The garment 310 includes a plurality of anchor structures 320 disposed on a first surface 312 of the garment 310 and a plurality of holes 330 penetrating the garment 310. The fabric electrodes 340 are disposed on the first surface 312 of the garment 310. The insulating film 350 is disposed on the first surface 312 of the garment 310 and seals peripherals of the fabric electrodes 340. The segment of the garment 310 without being covered by the insulating film 350 has a first elastic extension rate, the segment of the garment 310 being covered by the insulating film 350 has a second elastic extension rate, and the first elastic extension rate is greater than the second elastic extension rate.

In some embodiments, the distribution density of the holes 330 on the garment 310 can be varied according to different positions. For example, if a particular region 302 is observed having more deformation than other regions 304, the design of the garment 310 can be adjusted such that the region 302 may have more holes 330. That is, the distribution density of the holes 330 at the region 302 is greater than the distribution density of the holes 330 at the regions 304, to allow the wearable electromyography device 300 deforming smoothly. In some embodiments, the region 302 corresponds to the portion with large muscle amount.

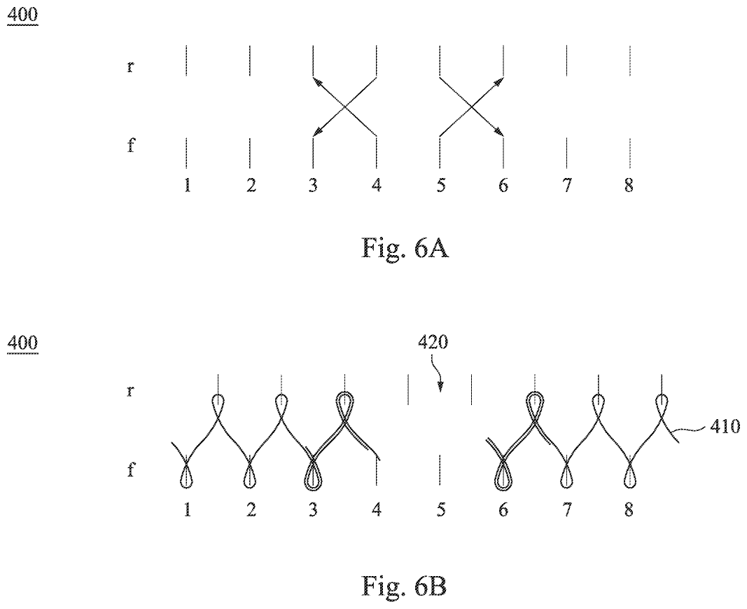

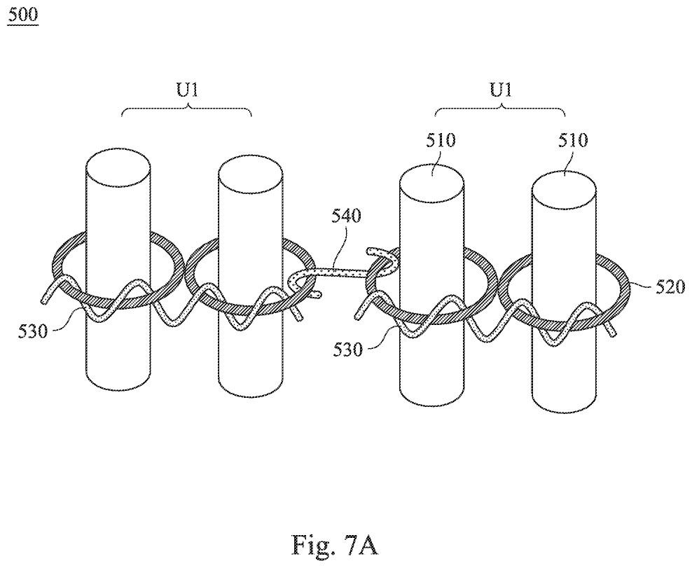

Reference is made to FIG. 6A and FIG. 6B, in which FIG. 6A is a knitting graph of the garment of the wearable electromyography device according to some embodiments of the invention, and FIG. 6B is a yarn loop schematic view of the garment of the wearable electromyography device according to some embodiments of the invention. In some embodiments, the garment 400 is a flat knitting fabric structure formed by knitting yarn loops provided by front needles and rear needles, in which the yarns 410 include friction yarns and elastic yarns. At the predetermined hole position, the yarn loops laterally shift one needle space thereby forming the hole 420. For example, if we want to form a hole 420 at the position between fourth needle and fifth needle, the yarn loop corresponding to the fourth needle is laterally shifted one needle space to the third needle, and the yarn loop corresponding to the fifth needle is laterally shifted one needle space to the sixth needle. The space between the fourth needle and the fifth needle is revealed and becomes the hole 420.

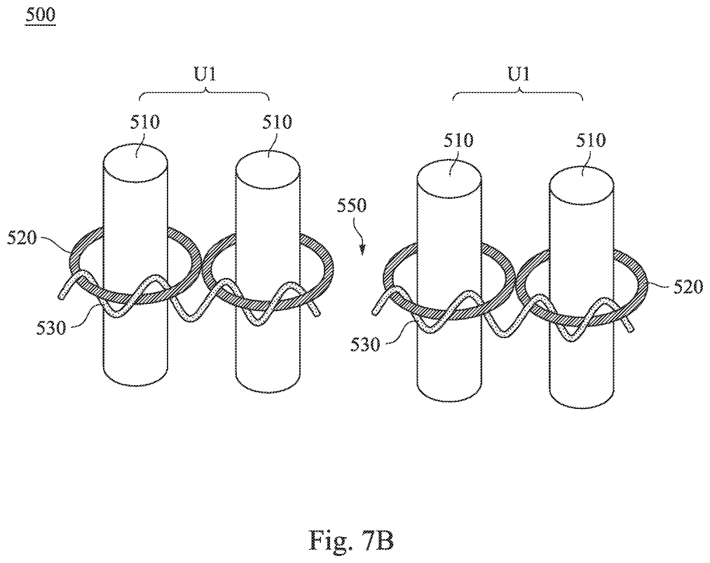

Reference is made to FIG. 7A and FIG. 7B, which are knitting graphs of the garment of the wearable electromyography device according to some embodiments of the invention. In some other embodiments, the garment 500 is a fabric structure formed by warp knitting. Each of the basic units U1 of the garment 500 includes two elastic yarns 510 parallel arranged in pair as warp yarns, and the friction yarns 520 are wound on the surface of the elastic yarns 510. The extensibility of the elastic yarns 510 is better than the extensibility of the friction yarns 520. The static friction coefficient of the friction yarns 520 is greater than the static friction coefficient of the elastic yarns 510. In some embodiments, the diameter of the elastic yarns 510 is greater than the diameter of the friction yarns 520. In some embodiments, the elastic yarns 510 can be rubber yarns.

The floating weft yarns 530 are wrapped on the friction yarns 520, and the floating weft yarns 530 only wrapped on the outer facing side of the friction yarns 520. Namely, the inner facing side of the friction yarns 520 is free of being wrapped by the floating weft yarns 530. Therefore, the friction yarns 520 are exposed at the inner facing side of the garment 500 to contact user's skin. The diameter of the floating weft yarns 530 is smaller than the diameter of the friction yarns 520. The loop density of the floating weft yarns 530 is greater than the loop density of the friction yarns 520. The section having the floating weft yarns 530 exposed at the outer facing side of the garment 500 can be served as the loops section of the loop and hook wrap fastener 290 in FIG. 4A.

Additionally, the adjacent basic units U1 are interconnected by linking yarns 540. More particularly, the linking yarns 540 selectively interconnect the basic units U1. As shown in FIG. 7A, the linking yarns 540 interconnect the friction yarns 520 of the adjacent basic units U1. As shown in FIG. 7B, the friction yarns 520 of the adjacent basic units U1 are not interconnected by the linking yarns 540 at the predetermined hole position thereby forming the hole 550 on the garment 500.

According to some embodiments of the invention, the wearable electromyography device is able to secure the electrodes at the correct positions thereby solving the problem of electrode shifting due to clothing falling or stretch deformation during the exercise. The accuracy of motion analysis using the wearable electromyography device can be improved accordingly.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A wearable electromyography device, comprising:
a garment comprising a plurality of anchor structures disposed on a first surface of the garment and a plurality of holes penetrating the garment;
a plurality of fabric electrodes disposed on the first surface of the garment; and
an insulating film disposed on the first surface of the garment and sealing peripherals of the fabric electrodes, wherein a segment of the garment without being covered by the insulating film has a first elastic extension rate, a segment of the garment being covered by the insulating film has a second elastic extension rate, and the first elastic extension rate is greater than the second elastic extension rate.

2. The wearable electromyography device of claim 1, wherein the first elastic extension rate is 130% to 160%.

3. The wearable electromyography device of claim 1, wherein the second elastic extension rate is 110% to 120%.

4. The wearable electromyography device of claim 1, wherein the holes are respectively disposed between the anchor structures.

5. The wearable electromyography device of claim 1, wherein the garment comprises a first region and a second region, and a distribution density of the holes at the first region is greater than a distribution density of the holes at the second region.

6. The wearable electromyography device of claim 1, further comprising a wrap fastener connected to the garment.

7. The wearable electromyography device of claim 6, wherein the wrap fastener comprises a loop and hook fastener.

8. The wearable electromyography device of claim 1, further comprising a plurality of conductive pieces disposed on the garment and connected to the fabric electrodes, respectively.

9. The wearable electromyography device of claim 8, further comprising a wireless transmission module connected to the conductive pieces.

10. The wearable electromyography device of claim 8, further comprising a plurality of fabric wirings connecting to the fabric electrodes to the conductive pieces, wherein the insulating film covers the fabric wirings.

11. The wearable electromyography device of claim 1, wherein the fabric electrodes comprise a first differential electrode pair, a second differential electrode pair, and a reference electrode.

12. The wearable electromyography device of claim 1, wherein a maximum static friction coefficient of the garment is from 0.45 to 0.50

13. The wearable electromyography device of claim 1, wherein an area ratio of the holes to the garment is from 10% to 15%.

14. The wearable electromyography device of claim 1, wherein the anchor structures comprise friction yarns knitted into the garment.

15. The wearable electromyography device of claim 14, wherein the friction yarns are knitted into the garment by flat knitting.

16. The wearable electromyography device of claim 14, wherein the friction yarns are knitted into the garment by warp knitting.

17. The wearable electromyography device of claim 16, wherein the garment comprises:
a plurality of basic units, each of the basic units comprising:
a plurality of elastic yarns;
a plurality of friction yarns wound on the elastic yarns; and
a plurality of floating weft yarns wrapped on an outer facing side of the friction yarns; and
a plurality of linking yarns selectively interconnecting the basic units.

18. The wearable electromyography device of claim 17, wherein the holes are spaces between the basic units without being interconnected by the linking yarns.

19. The wearable electromyography device of claim 17, wherein the linking yarns interconnect the friction yarns of the basic units.

20. The wearable electromyography device of claim 17, wherein an inner facing side of the friction yarns is free of being wrapped by the floating weft yarns.
